(19) United States
(12) Patent Application Publication
(10) Pub. No.: US 2011/0020470 A1
(43) Pub. Date: Jan. 27, 2011

(54) METHODS AND COMPOSITIONS FOR TREATING CANCER

(75) Inventors: Jonathan Kil, Seattle, WA (US); Eric Daniel Lynch, Lake Forest Park, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: SOUND PHARMACEUTICALS INCORPORATED, Seattle, WA (US)

(21) Appl. No.: 12/895,636

(22) Filed: Sep. 30, 2010

Related U.S. Application Data
(63) Continuation of application No. 11/371,186, filed on Mar. 8, 2006, now abandoned.
(60) Provisional application No. 60/661,429, filed on Mar. 8, 2005.

(51) Int. Cl. A61K 33/24; A61K 31/41; A61K 31/519; A61P 35/00

(52) U.S. Cl. 424/649; 514/359; 514/262.1

(57) ABSTRACT

In one aspect the present invention provides methods for treating cancer in a mammal, including the step of administering to a mammal suffering from a cancer an amount of ebselen that is sufficient to inhibit the growth of the cancer. In another aspect, the present invention provides methods for enhancing the chemotherapeutic effect of a platinum-containing chemotherapeutic agent administered to a mammal suffering from cancer.

### CROSS-REFERENCE TO RELATED APPLICATION

This application is a continuation of application Ser. No. 11/371,186, filed Mar. 8, 2006, which claims the benefit of Provisional Application No. 60/661,429, filed Mar. 8, 2005, both of which are herein incorporated by reference.

### FIELD OF THE INVENTION

The present invention relates to the use of ebselen, or a combination of ebselen and allopurinol, in chemotherapy for the treatment of cancer, and to methods for enhancing the chemotherapeutic effect of a platinum-containing chemotherapeutic agent, such as cisplatin.

### BACKGROUND OF THE INVENTION

One approach to the treatment of cancer is chemotherapy in which one or more chemical substances that are toxic, or otherwise deleterious, to the cancerous cells are administered to an individual suffering from cancer. Unfortunately, most, if not all, chemotherapeutic agents cause undesirable effects that adversely affect the health of the patient.

By way of example, the chemotherapeutic agent cisplatin (cis-diaminedichloroplatinum) is a heavy metal complex, with platinum as the central atom surrounded by two chloride atoms and two ammonia molecules in the cis position. Cisplatin produces interstrand and intrastrand crosslinkage in DNA of rapidly dividing cells, thus preventing DNA, RNA, and/or protein synthesis.

Cisplatin is typically used (often in combination with other chemotherapeutic agents, such as paclitaxel, cyclophosphomide, vinblastine, doxorubicin and bleomycin) to treat patients having metastatic testicular tumors, metastatic ovarian tumors, carcinoma of the endometrium, bladder, head, or neck. The anti-tumor activity of cisplatin against solid tumors such as breast and ovarian cancer has been well established. Unfortunately, cisplatin causes numerous adverse effects, such as seizures, peripheral neuropathies, ototoxicity, hearing loss, deafness, vertigo, dizziness, blurred vision, nausea, vomiting, anorexia, diarrhea, constipation, myelosuppression, thrombocytopenia, anemia, neutropenia, hepatotoxicity, and nephrotoxicity (see Yoshida, M. et al., Tohoku J. Exp. Med., 191:209-220, 2000; Baldew, G. S. et al., Cancer Res., 50:7031-7036, 1990; and Huang et al., Int. J. Dev. Neurosci., 18:259-270, 2000). The side effects of cisplatin, and other platinum-containing chemotherapeutic agents, can be so severe that it is not possible to administer the chemotherapeutic agent(s) to a patient for an extended time period.

With regard to cisplatin-related ototoxicity, studies in patients receiving cisplatin indicated that up to 90% will experience significant hearing loss, and that these changes are irreversible and cumulative (see Helson, L., Clin. Toxicol., 13:469-478, 1978). Characterization of cisplatin-related ototoxicity has revealed an increase in free radicals or reactive oxygen and nitrogen species such as the superoxide anion (O₂⁻) and nitric oxide (NO) related to cochlear injury. In particular, peroxynitrite (OONO⁻), a superoxide anion and nitric oxide, causes lipid peroxidation, a process that injures hair cell membranes (see Ryback, L. P., et al., Am. J. Otol., 21:513-520, 2000; Lynch et al., Anti-Cancer Drugs, 16:569-579, 2005). Cisplatin exposure has also been associated with a change in the level of reduced glutathione. The activity of glutathion utilizing enzymes has been correlated with outer hair cell loss due to cisplatin exposure (Ravi, R., et al., Pharmacol. Toxicol., 76:386-394, 1995; Lautermann, J., et al., Hear Res., 114:75-82, 1997; Rybak, L. P., et al., Laryngoscope, 109:1740-1744, 1999). Cisplatin exposure has also been shown to increase xanthine oxidase (XO) activity in the kidney (see Sogut, S., et al., Cell Biochem. Funct., 22:157-162, 2004). Studies involving carboplatin exposure show a similar increase in XO activity in the cochlea (see Husain, K., et al., Hear Res., 159:14-22, 2001). Although progress has been made in determining the biochemical mechanisms of cisplatin-related toxicity, no chemoprotective products have been developed that are effective in reducing cisplatin-associated oto- and nephrotoxicity.

Ovarian cancer is the fifth leading cause of cancer-related death (Barnes et al., Cancer J. Clin., 52:216-25, 2002). The treatment for ovarian cancer has evolved from the use of alkylating agents to platinum-based chemotherapy (e.g., cisplatin, carboplatin) in combination with taxane compounds (e.g., paclitaxel, docetaxel) (see Smith et al., Gynecologic Oncology, 98:141-145, 2005). Platinum-based chemotherapy is hampered by the dose-limiting cisplatin-related toxicity (e.g., neurotoxicity and nephrotoxicity) and carboplatin-related toxicity (e.g., myelosuppression, nephrotoxicity, and ototoxicity), as described above. While the taxanes have demonstrated activity in clinical studies for the treatment of numerous solid tumors, dose-limiting paclitaxel-related toxicity (e.g., peripheral neuropathies) or docetaxel-related toxicity (e.g., neurotoxicity, nephrotoxicity and myelosuppression) has also been observed (see, e.g., Smith et al., Gynecologic Oncology, 98:141-145, 2005). The additive neurotoxicity associated with cisplatin and paclitaxel has limited the tolerability of this treatment combination in the clinical setting. Id.

Thus, there is a need for chemotherapeutic compositions and methods that do not cause severely adverse effects when administered to a cancer patient, and that can, therefore, be administered to a patient over an extended period of time. In addition, there is a need for compositions and methods that enhance the chemotherapeutic effect of platinum-containing chemotherapeutic agents, such that a lower effective dosage of the chemotherapeutic agent can be used. In particular, there is a need for compositions and methods that enhance the chemotherapeutic effect of platinum-containing chemotherapeutic agents, and that also ameliorate or eliminate the undesirable effects of chemotherapy.

### SUMMARY OF THE INVENTION

The present inventors have discovered that ebselen, and the combination of ebselen and allopurinol, possesses chemotherapeutic activity. Thus, in one aspect, the present invention provides methods for treating cancer in a mammal. In one embodiment, the methods of this aspect of the invention include the step of administering to a mammal suffering from a cancer an amount of ebselen that is sufficient to inhibit the growth of the cancer. In another embodiment, the methods of this aspect of the invention include the step of administering to a mammal suffering from a cancer an amount of ebselen and an amount of allopurinol that together are sufficient to inhibit the growth of the cancer.

The present inventors have also discovered that ebselen, and the combination of ebselen and allopurinol, enhances the chemotherapeutic effect of platinum-containing chemotherapeutic agents. Thus, in another aspect, the present invention provides methods for enhancing the chemotherapeutic effect of a platinum-containing chemotherapeutic agent administered to a mammal suffering from cancer. In one embodiment, the methods of this aspect of the invention include the step of administering to a mammal suffering from cancer an amount of 2-phenyl-1,2-benzoiso selenazol-3(2H)-one (also called ebselen), that is sufficient to enhance the chemotherapeutic effect of a platinum-containing chemotherapeutic agent on the cancer, wherein the 2-phenyl-1,2-benzoisoselenazol-3(2H)-one is administered to the mammal before, during or after administration of the chemotherapeutic agent to the mammal. In another embodiment of this aspect of the invention, the methods include the step of administering to a mammal suffering from cancer an amount of allopurinol and an amount of ebselen that together are sufficient to enhance the chemotherapeutic effect of a platinum-containing chemotherapeutic agent on the cancer, wherein the allopurinol and the ebselen are administered to the mammal before, during or after administration of the chemotherapeutic agent to the mammal.

Additionally, the present inventors have discovered that the combination of ebselen and allopurinol ameliorates at least one adverse effect of chemotherapy. Thus, in another aspect, the present invention provides methods of ameliorating at least one adverse effect of a platinum-containing chemotherapeutic agent. The methods according to this aspect of the invention include the step of administering to a mammal suffering from cancer an amount of allopurinol and an amount of ebselen sufficient to ameliorate at least one adverse effect of the platinum-containing chemotherapeutic agent, wherein the allopurinol and ebselen are administered to the mammal before, during or after administration of the chemotherapeutic agent to the mammal. The methods of the invention are applicable to any mammal, such as a human being.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 shows a plot of the percentage of live, cultured, NuTu-19 ovarian cancer cells versus concentration of cisplatin in the culture medium, as described in Example 1. The number of live cells was measured after culturing the cells for 24 hours in the presence of cisplatin;

FIG. 2 shows a plot of the percentage of live, cultured, NuTu-19 ovarian cancer cells versus the concentration of ebselen in the culture medium, as described in Example 1. The viability of NuTu-19 cells cultured in the presence of ebselen, but not in the presence of cisplatin, is shown by the upper graph. The viability of NuTu-19 cells cultured in the presence of both ebselen and cisplatin (at a concentration of 43 μM) is shown by the lower graph;

FIG. 3 shows a plot of the percentage of live, cultured, NuTu-19 ovarian cancer cells versus the concentration of allopurinol in the culture medium, as described in Example 1. The viability of NuTu-19 cells cultured in the presence of allopurinol, but not in the presence of cisplatin, is shown by the upper graph. The viability of NuTu-19 cells cultured in the presence of both allopurinol and cisplatin (at a concentration of 43 μM) is shown by the lower graph;

FIG. 4 shows a plot of the percentage of live, cultured, NuTu-19 ovarian cancer cells versus the concentration of allopurinol in the culture medium, as described in Example 1. The viability of NuTu-19 cells cultured in the presence of allopurinol and ebselen (at a concentration of 47 μM), but not in the presence of cisplatin, is shown by the upper graph. The viability of NuTu-19 cells cultured in the presence of allopurinol and ebselen (at a concentration of 47 μM) and cisplatin (at a concentration of 43 μM) is shown by the lower graph;

FIG. 5 shows a graph showing the number of inner ear hair cells in rat cochlea that were cultured, in vitro, in the presence of 43 μM cisplatin (10), or 43 μM cisplatin plus 47 μM ebselen (12), or 47 μM ebselen (14), as described in Example 2;

FIG. 6 shows the permanent threshold shift (PTS) in hearing at 8 kHz, 16 kHz, 24 kHz and 32 kHz of rats treated with saline and DMSO (vehicle control) (20), or with cisplatin (at a dosage of 16 mg/kg body weight) in the presence of ebselen (at a dosage of 16 mg/kg body weight) (22), as described in Example 3;

FIG. 7 shows the permanent threshold shift (PTS) in hearing at 8 kHz, 16 kHz, 24 kHz and 32 kHz of rats treated with cisplatin (at a dosage of 16 mg/kg body weight) in the presence of allopurinol (at a dosage of 16 mg/kg body weight) (30), or in the presence of the combination of allopurinol (at a dosage of 8 mg/kg body weight) and ebselen (at a dosage of 8 mg/kg body weight) (32), as described in Example 3;

FIG. 8A shows the percentage of missing cochlear outer hair cells plotted against the distance from the apex of the cochlea in the left cochlea of a rat treated with the combination of cisplatin, saline and DMSO, as described in Example 3;

FIG. 8B shows the percentage of missing cochlear outer hair cells plotted against the distance from the apex of the cochlea in the left cochlea of a rat treated with the combination of cisplatin and ebselen, as described in Example 3;

FIG. 9A shows a plot of the percentage of live, cultured human ES-2 clear cell carcinoma ovarian cancer cells versus the concentration of ebselen. The ES-2 cells were cultured in the presence of either ebselen alone, cisplatin (4 μM) and paclitaxel (7.2 nM), or the combination of ebselen, cisplatin (4 μM), and paclitaxel (7.2 nM) as described in Example 5;

FIG. 9B shows a plot of the percentage of live, cultured human ES-2 clear cell carcinoma ovarian cancer cells versus the concentration of allopurinol. The ES-2 cells were cultured in the presence of either allopurinol alone, cisplatin (4 μM) and paclitaxel (7.2 nM), or the combination of allopurinol, cisplatin (4 μM), and paclitaxel (7.2 nM), as described in Example 5;

FIG. 9C shows a plot of the percentage of live, cultured human ES-2 clear cell carcinoma ovarian cancer cells versus the concentration of ebselen and allopurinol. The ES-2 cells were cultured in the presence of either ebselen and allopurinol, cisplatin (4 μM) and paclitaxel (7.2 nM), or the combination of ebselen, allopurinol, cisplatin (4 μM), and paclitaxel (7.2 nM), as described in Example 5;

FIG. 10A shows a plot of the percentage of live, cultured human SKOV-3 adenocarcinoma ovarian cancer cells versus the concentration of ebselen. The SKOV-3 cells were cultured in the presence of either ebselen, cisplatin (4.4 μM) and paclitaxel (10 nM), or the combination of ebselen, cisplatin (4.4 μM) and paclitaxel (10 nM), as described in Example 5;

FIG. 10B shows a plot of the percentage of live, cultured human SKOV-3 adenocarcinoma ovarian cancer cells versus the concentration of allopurinol. The SKOV-3 cells were cultured in the presence of either allopurinol, cisplatin (4.4 μM) and paclitaxel (10 nM), or the combination of allopurinol, cisplatin (4.4 μM), and paclitaxel (10 nM), as described in Example 5;

FIG. 10C shows a plot of the percentage of live, cultured human SKOV-3 adenocarcinoma ovarian cancer cells versus the concentration of ebselen and allopurinol. The SKOV-3 cells were cultured in the presence of either ebselen and allopurinol, cisplatin (4.4 μM) and paclitaxel (10 nM), or the combination of ebselen, allopurinol, cisplatin (4.4 μM), and paclitaxel (10 nM), as described in Example 5;

FIG. 11A shows a plot of the percentage of live, cultured human OVCAR-3 adenocarcinoma ovarian cancer cells versus the concentration of ebselen. The OVCAR-3 cells were cultured in the presence of either ebselen, cisplatin (1.4 μM) and paclitaxel (1.8 nM), or the combination of ebselen, cisplatin (1.4 μM), and paclitaxel (1.8 nM), as described in Example 5;

FIG. 11B shows a plot of the percentage of live, cultured human OVCAR-3 adenocarcinoma ovarian cancer cells versus the concentration of allopurinol. The OVCAR-3 cells were cultured in the presence of either allopurinol, cisplatin (1.4 μM) and paclitaxel (1.8 nM), or the combination of allopurinol, cisplatin (1.4 μM), and paclitaxel (1.8 nM), as described in Example 5;

FIG. 11C shows a plot of the percentage of live, cultured human OVCAR-3 adenocarcinoma ovarian cancer cells versus the concentration of ebselen and allopurinol. The OVCAR-3 cells were cultured in the presence of either ebselen and allopurinol, cisplatin (1.4 μM) and paclitaxel (1.8 nM), or the combination of ebselen, allopurinol, cisplatin (1.4 μM), and paclitaxel (1.8 nM), as described in Example 5;

FIG. 12A shows a plot of the percentage of live, cultured human CAOV-3 adenocarcinoma ovarian cancer cells versus the concentration of ebselen. The CAOV-3 cells were cultured in the presence of either ebselen, cisplatin (1.4 μM) and paclitaxel (1.76 nM), or the combination of ebselen, cisplatin (1.4 μM), and paclitaxel (1.76 nM), as described in Example 5;

FIG. 12B shows a plot of the percentage of live, cultured human CAOV-3 adenocarcinoma ovarian cancer cells versus the concentration of allopurinol. The CAOV-3 cells were cultured in the presence of either allopurinol, cisplatin (1.4 μM) and paclitaxel (1.76 nM), or the combination of allopurinol, cisplatin (1.4 μM), and paclitaxel (1.76 nM), as described in Example 5;

FIG. 12C shows a plot of the percentage of live, cultured human CAOV-3 adenocarcinoma ovarian cancer cells versus the concentration of ebselen and allopurinol. The CAOV-3 cells were cultured in the presence of either ebselen and allopurinol, cisplatin (1.4 μM) and paclitaxel (1.76 nM), or the combination of ebselen, allopurinol, cisplatin (1.4 μM), and paclitaxel (1.76 nM), as described in Example 5;

FIG. 13A shows a plot of the percentage of live, cultured human OV-90 papillary serous adenocarcinoma ovarian cancer cells versus the concentration of ebselen. The OV-90 cells were cultured in the presence of either ebselen, cisplatin (4.4 μM) and paclitaxel (38.5 nM), or the combination of ebselen, cisplatin (4.4 μM), and paclitaxel (38.5 nM), as described in Example 5;

FIG. 13B shows a plot of the percentage of live, cultured human OV-90 papillary serous adenocarcinoma ovarian cancer cells versus the concentration of allopurinol. The OV-90 cells were cultured in the presence of either allopurinol, cisplatin (4.4 μM) and paclitaxel (38.5 nM), or the combination of allopurinol, cisplatin (4.4 μM), and paclitaxel (38.5 nM), as described in Example 5;

FIG. 13C shows a plot of the percentage of live, cultured human OV-90 papillary serous adenocarcinoma ovarian cancer cells versus the concentration of ebselen and allopurinol. The OV-90 cells were cultured in the presence of either ebselen and allopurinol, cisplatin (4.4 μM) and paclitaxel (38.5 nM), or the combination of ebselen, allopurinol, cisplatin (4.4 μM), and paclitaxel (38.5 nM), as described in Example 5;

FIG. 14A shows a plot of the percentage of live, cultured human TOV-112D adenocarcinoma/endometroid carcinoma ovarian cancer cells versus the concentration of ebselen. The TOV-112D cells were cultured in the presence of either ebselen, cisplatin (1.05 μM) and paclitaxel (2.6 nM), or the combination of ebselen, cisplatin (1.05 μM), and paclitaxel (2.6 nM), as described in Example 5;

FIG. 14B shows a plot of the percentage of live, cultured human TOV-112D adenocarcinoma/endometroid carcinoma ovarian cancer cells versus the concentration of allopurinol. The TOV-112D cells were cultured in the presence of either allopurinol, cisplatin (1.05 μM) and paclitaxel (2.6 nM), or the combination of allopurinol, cisplatin (1.05 μM), and paclitaxel (2.6 nM), as described in Example 5;

FIG. 14C shows a plot of the percentage of live, cultured human TOV-112D adenocarcinoma/endometroid carcinoma ovarian cancer cells versus the concentration of ebselen and allopurinol. The TOV-112D cells were cultured in the presence of either ebselen and allopurinol, cisplatin (1.05 μM) and paclitaxel (2.6 nM), or the combination of ebselen, allopurinol, cisplatin (1.05 μM), and paclitaxel (2.6 nM), as described in Example 5;

FIG. 15A shows a plot of the percentage of live, cultured human TOV-21G adenocarcinoma/clear cell carcinoma ovarian cancer cells versus the concentration of ebselen. The TOV-21G cells were cultured in the presence of either ebselen, cisplatin (4.8 μM) and paclitaxel (80 nM), or the combination of ebselen, cisplatin (4.8 μM), and paclitaxel (80 nM), as described in Example 5;

FIG. 15B shows a plot of the percentage of live, cultured human TOV-21G adenocarcinoma/clear cell carcinoma ovarian cancer cells versus the concentration of allopurinol. The TOV-21G cells were cultured in the presence of either allopurinol, cisplatin (4.8 μM) and paclitaxel (80 nM), or the combination of allopurinol, cisplatin (4.8 μM), and paclitaxel (80 nM), as described in Example 5;

FIG. 15C shows a plot of the percentage of live, cultured human TOV-21G adenocarcinoma/clear cell carcinoma ovarian cancer cells versus the concentration of ebselen and allopurinol. The TOV-21G cells were cultured in the presence of either ebselen and allopurinol, cisplatin (4.8 μM) and paclitaxel (80 nM), or the combination of ebselen, allopurinol, cisplatin (4.8 μM), and paclitaxel (80 nM), as described in Example 5;

FIG. 16A shows a plot of the percentage of live, cultured rat rSPI-tu epithelial ovarian cancer cells versus the concentration of ebselen. The rSPI-tu cells were cultured in the presence of either ebselen, cisplatin (1.5 μM) and paclitaxel (90 nM), or the combination of ebselen, cisplatin (1.5 μM), and paclitaxel (90 nM), as described in Example 5;

FIG. 16B shows a plot of the percentage of live, cultured rat rSPI-tu epithelial ovarian cancer cells versus the concentration of allopurinol. The rSPI-tu cells were cultured in the presence of either allopurinol, cisplatin (1.5 μM) and paclitaxel (90 nM), or the combination of allopurinol, cisplatin (1.5 μM), and paclitaxel (90 nM), as described in Example 5; and

FIG. 16C shows a plot of the percentage of live, cultured rat rSPI-tu epithelial ovarian cancer cells versus the concentration of ebselen and allopurinol. The rSPI cells were cultured in the presence of either ebselen and allopurinol, cisplatin (1.5 μM) and paclitaxel (90 nM), or the combination of ebselen, allopurinol, cisplatin (1.5 μM), and paclitaxel (90 nM), as described in Example 5.

### DETAILED DESCRIPTION OF THE PREFERRED EMBODIMENT

As used herein, the term “ameliorating at least one adverse effect of chemotherapy” includes: (a) reducing the magnitude and/or duration of at least one adverse effect of chemotherapy; and/or (b) completely eliminating at least one adverse effect of chemotherapy; and/or (c) preventing the onset of one or more adverse effect(s) of chemotherapy that would occur without administration of the combination of ebselen and allopurinol.

As used herein, the term “chemotherapeutic agent” is an agent that is administered to a mammalian subject to kill, or otherwise adversely affect, cancer cells (e.g., completely or partially inhibit the growth of cancer cells).

As used herein, the term “enhancing the chemotherapeutic effect of a platinum-containing chemotherapeutic agent” includes enhancing the ability of a platinum-containing chemotherapeutic agent to kill cancer cells and/or to slow the rate of growth or cell division of cancer cells when administered to a mammal suffering from cancer.

The present inventors have found that ebselen and the combination of ebselen and allopurinol possesses chemotherapeutic activity when administered to a mammal suffering from cancer. Thus, in one aspect, the present invention provides methods for treating cancer in a mammal. In one embodiment, the methods of this aspect of the invention include the step of administering to a mammal suffering from a cancer an amount of ebselen that is sufficient to inhibit the growth of the cancer. In another embodiment, the methods of this aspect of the invention include the step of administering to a mammal suffering from a cancer an amount of ebselen and an amount of allopurinol that together are sufficient to inhibit the growth of the cancer. The methods of the invention are applicable to any mammal, such as a human being.

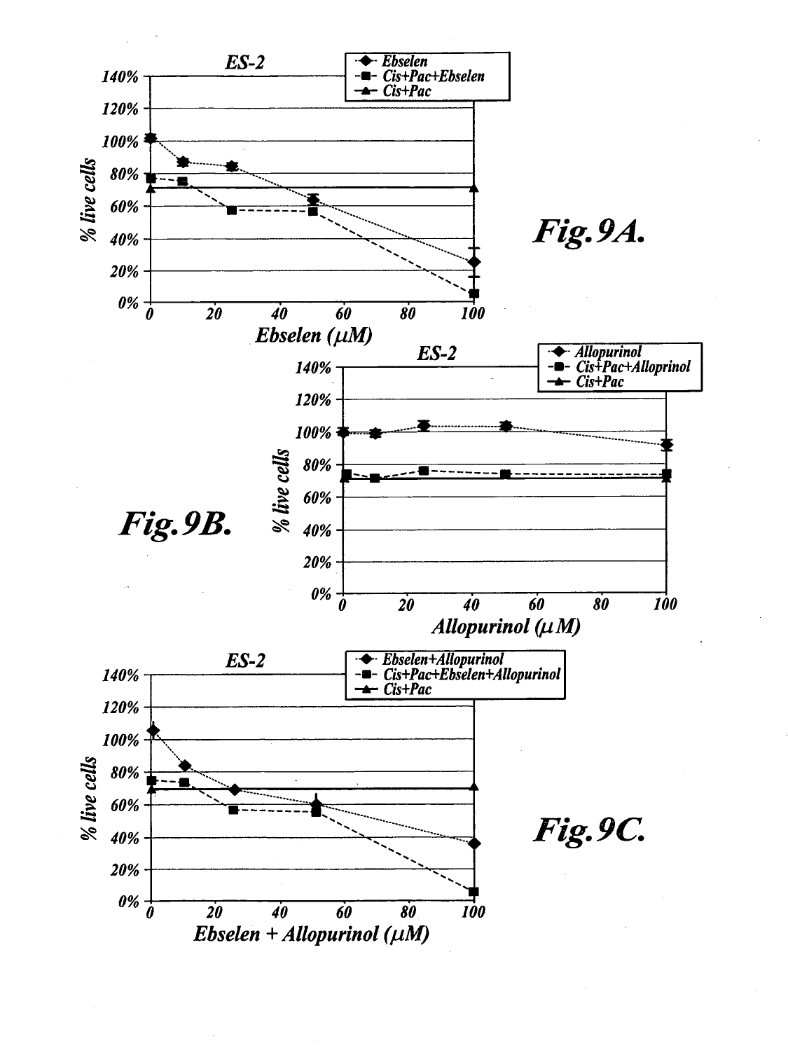

The present inventors have found that ebselen and the combination of ebselen and allopurinol possesses chemotherapeutic activity when contacted with tumor cell lines, as described in Example 5 and shown in Table 3, Table 4, and FIGS. 9A-16C. The methods of this aspect of the present invention are effective, for example, against cancers of the female reproductive system, such as ovarian cancer; testicular cancer; cancers of the head or neck, and cancers that exhibit multi-drug resistance. Ebselen, a seleno-organic compound, is known to have excellent oral availability, has been shown to be non-toxic in a non-cancer cell line (Baldew G S et al., Biochem Pharmacol 44(2): 382-7 (1992), and has been evaluated in human clinical testing for the treatment of acute ischemic stroke, where no adverse events were identified (see Fischer, H., et al., Xenobiotica, 18:1347-1359, 1988; Yamaguchi, T., et al., Stroke, 29:12-17, 1998; and Ogawa, A., et al., Cerebrovasc. Dis. 9:112-118, 1999).

Unless stated otherwise, any isomeric or tautomeric form of allopurinol and 2-phenyl-1,2-benzoisoselenazol-3(2H)-one can be used in the invention. Any pharmaceutically acceptable salt of allopurinol and 2-phenyl-1,2-benzoisoselenazol-3(2H)-one can be used in the invention.

Exemplary dosages for allopurinol are 10-2400 mg/day, such as 50-1200 mg/day, or such as 100-800 mg/day. Exemplary dosages for ebselen are 5-5000 mg/day, such as 50-2000 mg/day, or such as 500-1000 mg/day. The abbreviation “mg” means milligrams.

An advantage of using ebselen or the combination of ebselen and allopurinol to treat cancer is that a mammalian subject suffering from cancer can be administered an amount of ebselen and an amount of allopurinol over an extended period of time that do not cause the highly deleterious, and potentially life-threatening, side effects caused by most other chemotherapeutic agents (e.g., damage to the vital organs and immune system). Thus, for example, a cancer patient can be treated with a traditional chemotherapeutic agent (e.g., cisplatin) for a limited time (e.g., periodic doses over a period of several weeks or months) in accordance with art-recognized dosage regimes for the chemotherapeutic agent(s) being used. Thereafter, the cancer patient can be periodically administered an amount of ebselen or the combination of allopurinol and ebselen that is effective to kill remaining cancer cells, or completely or partially inhibit the growth of remaining cancer cells, or partially inhibit the growth of new cancer cells. Ebselen, or the combination of allopurinol and ebselen can be administered over a period of several months or years, and the dosage can be selected to avoid causing substantial adverse side effects in the cancer patient when administered over an extended time period.

For example, if a cancer patient is administered a weekly dose of cisplatin once each week for four weeks, then ebselen or ebselen and allopurinol can be administered once per day for each day during the four-week period. The ebselen or ebselen and allopurinol can thereafter be administered daily or once per week for a period of from one month to 24 months after completion of the treatment with cisplatin. Exemplary dosages for allopurinol are 10-2400 mg/day, such as 50-1200 mg/day, or such as 100-800 mg/day. Exemplary dosages for ebselen are 5-5000 mg/day, such as 50-2000 mg/day, such as 500-1000 mg/day.

In another embodiment, the present invention provides methods for enhancing the chemotherapeutic effect of a platinum-containing chemotherapeutic agent administered to a mammal suffering from cancer, the method comprising the step of administering to a mammal suffering from cancer an amount of 2-phenyl-1,2-benzoisoselenazol-3(2H)-one (also called ebselen) that is sufficient to enhance the chemotherapeutic effect of a platinum-containing chemotherapeutic agent on the cancer, wherein the 2-phenyl-1,2-benzoisoselenazol-3(2H)-one is administered to the mammal before, during or after administration of the chemotherapeutic agent to the mammal.

In accordance with this embodiment, the mammal typically receives one dose of ebselen for each dose of chemotherapeutic agent(s). The ebselen may be administered to the mammal before, during, or after administration to the mammal of the platinum-containing chemotherapeutic agent, provided that administration of the ebselen occurs sufficiently close, in time, to the administration of the platinum-containing chemotherapeutic agent that the ebselen and platinum-containing chemotherapeutic agent are present together in the body of the mammalian subject for a sufficient period of time to permit the ebselen to enhance the chemotherapeutic effect of the platinum-containing chemotherapeutic agent.

In yet another embodiment, the present invention provides methods for enhancing the chemotherapeutic effect of a platinum-containing chemotherapeutic agent administered to a mammal suffering from cancer, the method comprising the step of administering to a mammal suffering from cancer an amount of allopurinol and an amount of ebselen that together are sufficient to enhance the chemotherapeutic effect of a platinum-containing chemotherapeutic agent on the cancer, where the allopurinol and the ebselen are administered to the mammal before, during or after administration of the chemotherapeutic agent to the mammal.

In accordance with this embodiment, the mammal typically receives one dose of ebselen and allopurinol for each dose of chemotherapeutic agent(s). The ebselen and allopurinol may be administered to the mammal before, during, or after administration to the mammal of the platinum-containing chemotherapeutic agent, provided that administration of the ebselen and allopurinol occurs sufficiently close, in time, to the administration of the platinum-containing chemotherapeutic agent that the ebselen, allopurinol and platinum-containing chemotherapeutic agent are all present together in the body of the mammalian subject for a sufficient period of time to permit the ebselen and allopurinol to enhance the chemotherapeutic effect of the platinum-containing chemotherapeutic agent. The ebselen may be administered separately from the allopurinol, or together with the allopurinol.

For example, in some embodiments of the invention, ebselen, or the combination of ebselen and allopurinol are administered to a mammalian subject at any time during a period extending from 18 hours before administration of one or more platinum-containing chemotherapeutic agents to the mammalian subject, to 18 hours after administration of one or more platinum-containing chemotherapeutic agents to the mammalian subject. In some embodiments of the invention, ebselen, or the combination of ebselen and allopurinol are administered to a mammalian subject at any time during a period extending from one hour before administration of one or more platinum-containing chemotherapeutic agents to the mammalian subject, to one hour after administration of one or more platinum-containing chemotherapeutic agents to the mammalian subject. In some embodiments of the invention, ebselen, or the combination of ebselen and allopurinol are administered to a mammalian subject at any time during a period extending from 10 minutes before administration of one or more platinum-containing chemotherapeutic agents to the mammalian subject, to ten minutes after administration of one or more chemotherapeutic agents to the mammalian subject. In some embodiments of the invention, ebselen, or the combination of ebselen and allopurinol are administered to a mammalian subject concurrently with administration of one or more platinum-containing chemotherapeutic agents to the mammalian subject.

The methods of the invention are applicable to any mammal, such as a human being, undergoing any form of chemotherapy that uses a platinum-containing chemotherapeutic agent. Examples of platinum-containing chemotherapeutic agents include cisplatin, carboplatin, and oxaliplatin.

In some embodiments of the method, the platinum-containing chemotherapeutic agents may be combined with one or more taxane-containing chemotherapeutic agents in the development of combination therapies. Taxane-containing agents are classified as anti-tubular agents that stabilize tubulin polymerization and cell arrest in the M and G2 phases of the cell cycle. Examples of taxane-containing chemotherapeutic agents include docetaxel and paclitaxel.

The methods of the present invention are effective, for example, against cancers of the female urogenital and reproductive system, such as ovarian, cervical, uterine and bladder cancers; prostate and testicular cancers; cancers of the head or neck; and, more generally, solid tumors that are epithelial or endothelial in origin (e.g., adenocarcinoma of the ovary).

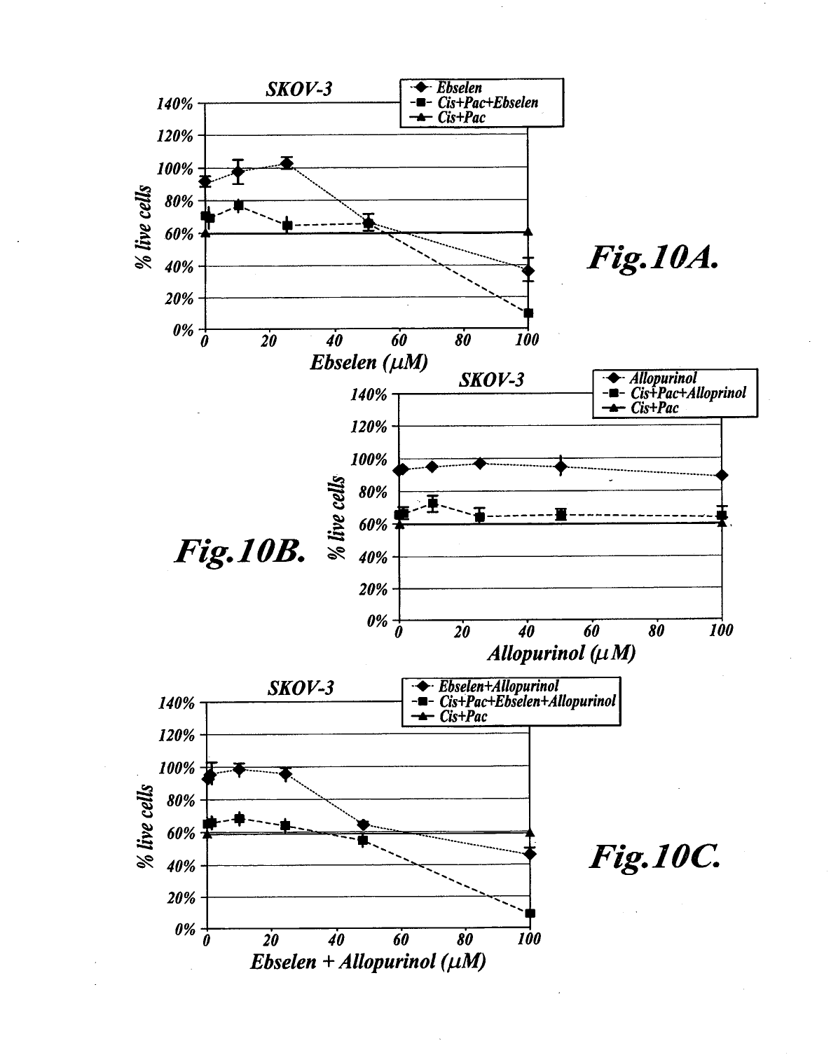

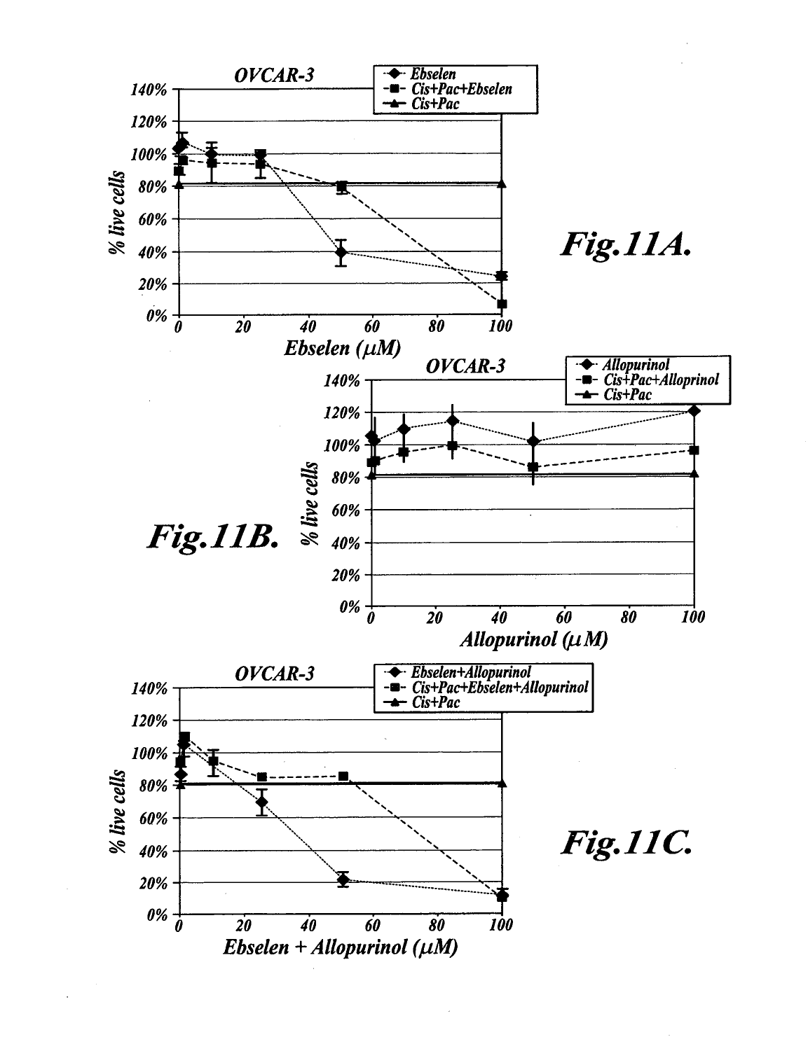

The methods of the present invention are also effective to enhance the chemotherapeutic effect of a platinum-containing chemotherapeutic agent against tumors that exhibit multi-drug chemotherapy resistance. The development of multi-drug resistance (MDR) is a major cause of failure of cancer chemotherapy. The MDR phenotype is characterized by resistance to a broad spectrum of cytotoxic drugs, including resistance to platinum-containing agents. MDR may be intrinsic (before exposure to chemotherapeutic agents) or may be acquired after chemotherapy. The over-expression of some ATP binding cassette (ABC) transporters has been linked with MDR (see Vanden Heuvel-Eibrink et al., Int. J. Clin. Pharm. and Ther., 38:94-110, 2000). The endogenous task of the ABC transporters is to transport a variety of different molecules across cell membranes, including amino acids, nucleotides, sugars, lipids, and peptides. Such transport is particularly problematic in tumor cells, where it interferes with therapeutic treatment of cancer due to the active transport of cytotoxic agents to the exterior of the cell membrane. Such tumor cells are called “multi-drug chemotherapy resistant” cells. The present inventors have shown that ebselen and the combination of ebselen and allopurinol enhance the cytotoxic activity of a platinum-containing chemotherapeutic agent against the human ovarian tumor cell lines ES-2 (see FIGS. 9A-C), SKOV-3 (see FIGS. 10A-C) and OVCAR-3 (see FIGS. 11A-11C), which are known to exhibit multi-drug chemotherapy resistance (see Smith, J. A., et al., Gynecologic Oncology, 98:141-145, 2005).

Unless stated otherwise, any isomeric or tautomeric form of allopurinol and 2-phenyl-1,2-benzoisoselenazol-3(2H)-one can be used in the invention. Any pharmaceutically acceptable salt of allopurinol and 2-phenyl-1,2-benzoisoselenazol-3(2H)-one can be used in the invention.

By way of example, the following representative allopurinol derivatives are useful in the practice of the invention to enhance the chemotherapeutic effect of a platinum-containing chemotherapeutic agent: 1-methylallopurinol; 2-methylallopurinol; 5-methylallopurinol; 7-methylallopurinol; 1,5-dimethylallopurinol; 2,5-dimethylallopurinol; 1,7-dimethylallopurinol; 2,7-dimethylallopurinol; 5,7-dimethylallopurinol; 2,5,7-trimethylallopurinol; 1-ethoxycarbonylallopurinol; and 1-ethoxycarbonyl-5-methylallopurinol.

Exemplary dosages for allopurinol are 10-2400 mg/day, such as 50-1200 mg/day, or such as 100-800 mg/day. Exemplary dosages for ebselen are 5-5000 mg/day, such as 50-2000 mg/day, or such as 500-1000 mg/day. The abbreviation “mg” means milligrams. At least one dose of ebselen, either alone or in combination with at least one dose of allopurinol, is administered to a mammalian subject for each dose of chemotherapeutic agent administered to the mammalian subject. Dosage regimes for chemotherapeutic agents are known in the art. The ability of ebselen alone, or the combination of ebselen and allopurinol to enhance the chemotherapeutic activity of a platinum-containing chemotherapeutic agent permits the administration of a lower effective dose of the platinum-containing chemotherapeutic agent when the chemotherapeutic agent is administered with ebselen alone, or with the combination of ebselen and allopurinol, as compared to when the chemotherapeutic agent is administered without ebselen or ebselen and allopurinol.

Thus, for example, conventional treatment of a human cancer patient with cisplatin can include three or four weekly doses of cisplatin administered intravenously at a dosage of 80 mg to 100 mg cisplatin per meter² patient body area. Cisplatin dosage can be, for example, as low as 25 mg/meter² patient body area when combined with either ebselen or the combination of ebselen and allopurinol. By way of example, in the practice of the present invention, a daily dose of at least 50 mg/day for ebselen, or the combination of at least 50 mg/day for ebselen and at least 50 mg/day for allopurinol, can be used in combination with a platinum-containing chemotherapeutic agent. For example, a daily dose of ebselen at 300 mg/day either alone or in combination with a daily dose of allopurinol at 300 mg/day can be used in combination with a platinum-containing agent.

Administration of the ebselen and allopurinol is accomplished by any effective route, e.g., orally or parenterally. Methods of parenteral delivery include topical, intra-arterial, subcutaneous, intramedullary, intravenous, or intranasal administration. The ebselen and allopurinol may be formulated with suitable pharmaceutically acceptable carriers comprising excipients and other compounds that facilitate administration of the ebselen and allopurinol to a mammalian subject undergoing chemotherapy. Further details on techniques for formulation and administration may be found in the latest edition of Remington's Pharmaceutical Sciences (Maack Publishing Co, Easton, Pa.).

Ebselen and allopurinol formulated for oral administration can be formulated using pharmaceutically acceptable carriers well known in the art, in dosages suitable for oral administration. Such carriers enable the ebselen and allopurinol to be formulated as tablets, pills, dragees, capsules, liquids, gels, syrups, slurries, suspensions, etc., suitable for ingestion by a mammalian subject.

A composition comprising ebselen or ebselen and allopurinol for oral use can be obtained, for example, through combination of ebselen or ebselen and allopurinol with solid excipient, optionally grinding the resulting mixture, and processing the mixture of granules, after adding suitable additional compounds, if desired, to obtain tablets or dragee cores. Suitable excipients are carbohydrate or protein fillers. These include, but are not limited to, sugars, including lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins, such as gelatin and collagen. If desired, disintegrating or solubilising agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage).

Compositions comprising ebselen or ebselen and allopurinol, which can be used orally, can be formulated, for example, as push-fit capsules made of gelatin, as well as soft, sealed capsules made of gelatin and a coating such as glycerol or sorbitol. Push-fit capsules can contain ebselen and allopurinol mixed with filler or binders such as lactose or starches, lubricants such as talc or magnesium stearate, and, optionally, stabilizers. In soft capsules, the ebselen and allopurinol may be dissolved or suspended in suitable liquids, such as fatty oils, liquid paraffin, or liquid polyethylene glycol with or without stabilizers.

Compositions comprising ebselen or ebselen and allopurinol for parenteral administration include aqueous solutions of ebselen and/or allopurinol. For injection, the composition comprising ebselen and/or allopurinol may be formulated in aqueous solutions, preferably in physiologically compatible buffers such as Hank's solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of ebselen and/or allopurinol may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents, which increase the solubility of the compounds to allow for the preparation of highly concentrated solutions.

For topical or nasal administration, penetrants appropriate to the particular barrier to be permeated are typically used in the formulation. Such penetrants are generally known in the art.

Compositions comprising ebselen or ebselen and allopurinol may be manufactured in a manner similar to that known in the art (e.g., by means of conventional mixing, dissolving, granulating, dragee-making, levigating, emulsifying, encapsulating, entrapping, or lyophilising processes). Compositions comprising ebselen or ebselen and allopurinol may also be modified to provide appropriate release characteristics, e.g., sustained release or targeted release, by conventional means (e.g., coating).

Ebselen and allopurinol may each be provided as a salt and can be formed with many acids, including but not limited to hydrochloric, sulfuric, acetic, lactic, tartaric, malic, succinic, etc. Salts tend to be more soluble in aqueous or other protonic solvents than are the corresponding free base forms.

The amounts of ebselen and allopurinol actually administered will be dependent upon the individual to which treatment is to be applied, and will preferably be an optimized amount such that the desired effect is achieved without significant side effects. The determination of an effective dose is well within the capability of those skilled in the art.

The present inventors have found that, in addition to enhancing the chemotherapeutic effect of platinum-containing chemotherapeutic agents, the combination of ebselen and allopurinol acts as a chemoprotectant that ameliorates some or all of the adverse effects of platinum-containing chemotherapeutic agents. Thus, in another embodiment, the present invention provides methods of ameliorating at least one adverse effect of a platinum-containing agent, the method comprising the step of administering to a mammal suffering from cancer an amount of allopurinol and an amount of ebselen sufficient to ameliorate at least one adverse effect of the platinum-containing agent. The principal adverse effects of platinum-containing chemotherapeutic agents are: nephrotoxicity, neurotoxicity, ototoxicity, myelosuppression, alopecia, weight loss, vomiting, nausea and immunosuppression.

The present inventors have discovered that ebeselen and allopurinol enhance the chemotherapeutic effect of platinum-containing chemotherapeutic agents (as described supra), and that ebselen and allopurinol also ameliorate or eliminate the undesirable effects of chemotherapy with platinum-containing chemotherapeutic agents. Example 3 herein describes the results of an experiment showing that the combination of allopurinol and ebselen protect rat inner ear cells from damage caused by the chemotherapeutic agent, cisplatin.

The following examples merely illustrate the best mode now contemplated for practicing the invention, but should not be construed to limit the invention. All literature citations herein are expressly incorporated by reference.

### Example 1

This example shows that ebselen and allopurinol, alone, or in combination, do not inhibit the ability of cisplatin to kill cultured NuTu-19 ovarian cancer tumor cells as measured using the MTS cell viability assay.

NuTu-19 cells were plated at a density of 3,000 cells per well in 96 well culture dishes, and incubated at 37° C., in the presence of 5% carbon dioxide, for 24 hours. N-acetylcysteine, ebselen or allopurinol were incubated for one hour, or for four hours, with the NuTu-19 cells, then cisplatin was added to the cultures, which were further incubated at 37° C., in the presence of 5% carbon dioxide, for 24 hours. The NuTu-19 cells were then rinsed with media and incubated in the presence of cisplatin for an additional 24 hours.

The NuTu-19 cells were then rinsed twice with phosphate buffered saline (PBS), then MTS assays were performed to measure the number of living cells. MTS is an abbreviation for (3-(4,5-dimethylthiazol-2-yl)-5-(3-carboxymethoxyphenyl)-2-(4-sulfophenyl)-2H-tetrazolium. The MTS assay is a colorimetric method for determining the number of viable cells based upon physiologic catabolism of MTS to a formazan product that is soluble in tissue culture medium. The absorbance of the formazan product at 490 nm can be measured directly from a 96 well plate using a plate reader. Increased absorbance at 490 nm correlates with increased production of formazan in a well. This is typically due to more viable cells present in a well.

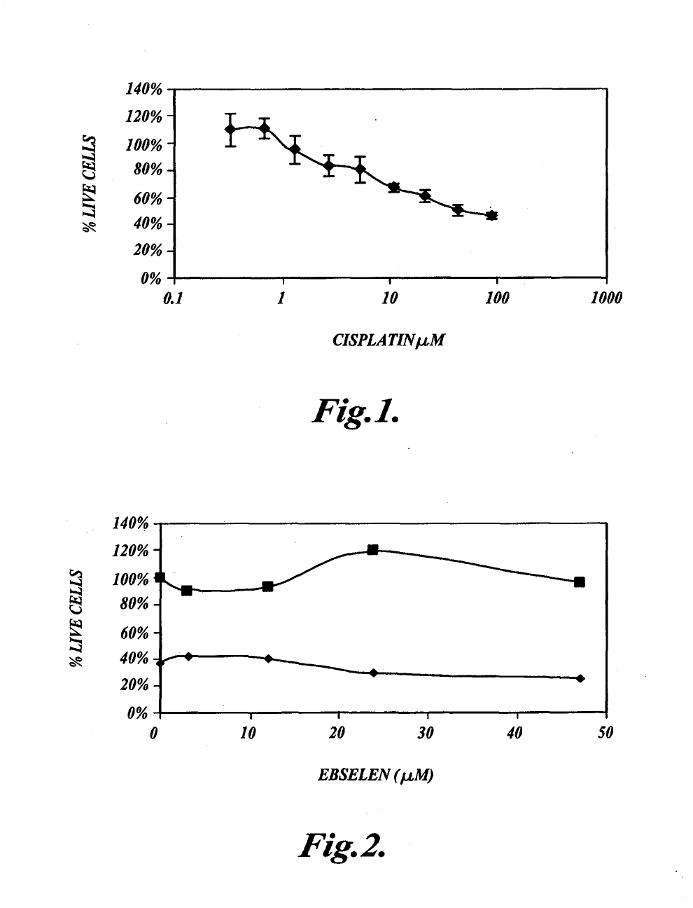

FIG. 1 shows a plot of the percentage of live, cultured, NuTu-19 ovarian cancer cells versus concentration of cisplatin in the culture medium. The data set forth in FIG. 1 show that cultured NuTu-19 ovarian cancer cells are killed after incubation for 24 hours in the presence of cisplatin.

FIG. 2 shows a plot of the percentage of live, cultured, NuTu-19 ovarian cancer cells versus the concentration of ebselen in the culture medium. The viability of NuTu-19 cells cultured in the presence of ebselen, but not in the presence of cisplatin, is shown by the upper graph. The viability of NuTu-19 cells cultured in the presence of both ebselen and cisplatin (at a concentration of 43 μM) is shown by the lower graph. The data set forth in FIG. 2 shows that ebselen does not inhibit the ability of cisplatin to kill NuTu-19 ovarian cancer tumor cells in culture.

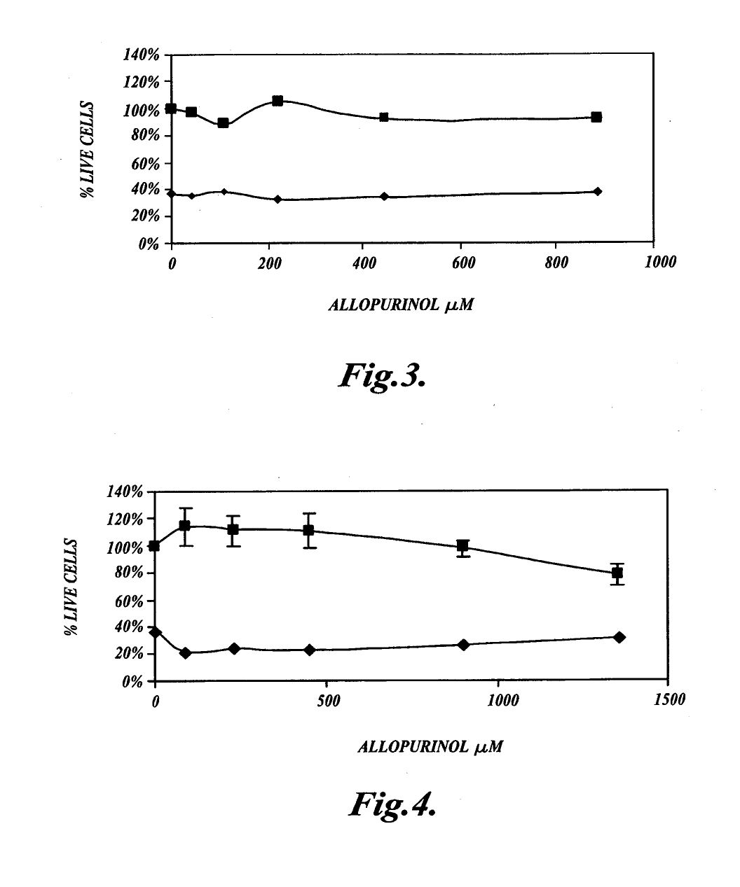

FIG. 3 shows a plot of the percentage of live, cultured, NuTu-19 ovarian cancer cells versus the concentration of allopurinol in the culture medium. The viability of NuTu-19 cells cultured in the presence of allopurinol, but not in the presence of cisplatin, is shown by the upper graph. The viability of NuTu-19 cells cultured in the presence of both allopurinol and cisplatin (at a concentration of 43 μM) is shown by the lower graph. The data set forth in FIG. 3 shows that allopurinol does not inhibit the ability of cisplatin to kill NuTu-19 ovarian cancer tumor cells in culture.

FIG. 4 shows a plot of the percentage of live, cultured, NuTu-19 ovarian cancer cells versus the concentration of allopurinol in the culture medium. The viability of NuTu-19 cells cultured in the presence of allopurinol and ebselen (at a concentration of 47 μM), but not in the presence of cisplatin, is shown by the upper graph. The viability of NuTu-19 cells cultured in the presence of allopurinol and ebselen (at a concentration of 47 μM) and cisplatin (at a concentration of 43 μM) is shown by the lower graph. The data set forth in FIG. 4 shows that the combination of allopurinol and ebselen does not inhibit the ability of cisplatin to kill NuTu-19 ovarian cancer tumor cells in culture.

### Example 2

This Example shows that ebselen protects inner ear hair cells from damage by cisplatin in vitro.

Three cochlea per treatment, obtained from P3-4 mouse pups, were cultured in 0.4 micrometer MilliCell-CM inserts with NeuroBasal A medium plus B27 supplement. After 24 hours in culture, ebselen was added to the medium, incubated for ten minutes, and then cisplatin was added to the medium at a final concentration of 43 μM. A first control treatment included 43 μM cisplatin. A second control treatment included 47 μM ebselen without the addition of cisplatin. All cultures were incubated for 24 hours at 37° C. in 5% carbon dioxide.

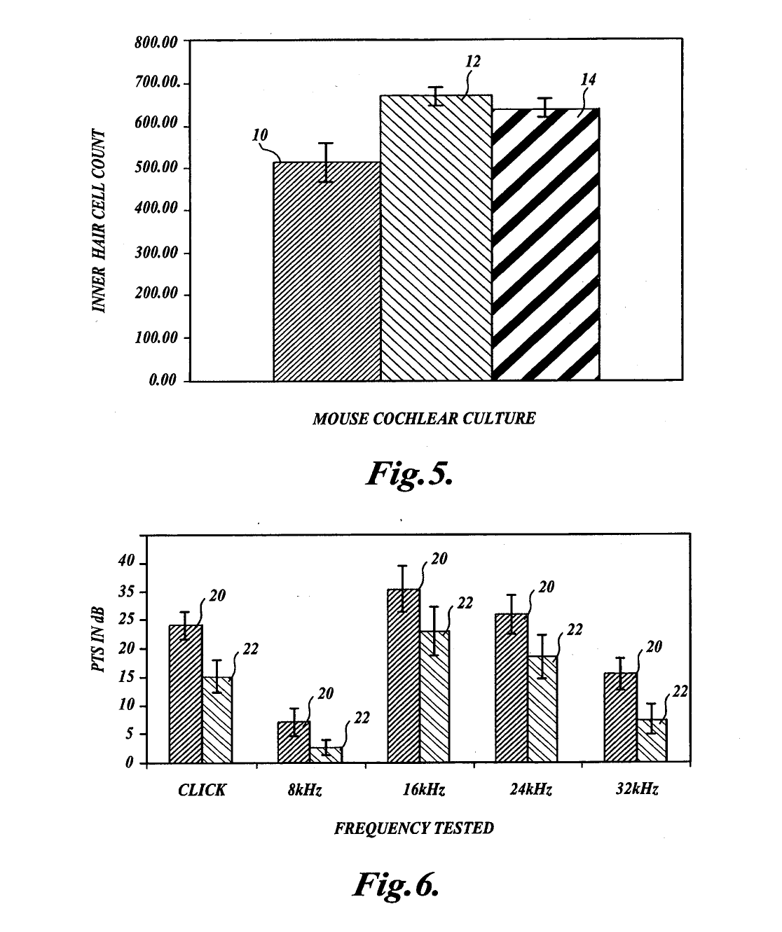

The explants were then harvested, fixed, and stained with calbindin (which detects hair cells) and DAPI (4′,6-Diamindino-2-phenylindole; for detection of nuclear DNA). FIG. 5 shows the number of inner ear hair cells in mice cochlea that were cultured, in vitro, in the presence of 43 μM cisplatin (10), or 43 μM cisplatin plus 47 μM ebselen (12), or 47 μM ebselen (14). The data set forth in FIG. 5 shows that ebselen protects inner ear hair cells from damage by cisplatin in vitro.

The concentrations of cisplatin and ebselen used in the experiments described in this Example are the same concentrations of cisplatin and ebselen that were used in the cell culture assays described in Example 1. Thus, the experiments reported in Example 1 and Example 2 together show that, at the concentration utilized in these experiments, ebselen does not protect NuTu-19 ovarian cancer tumor cells from the toxic effects of cisplatin, but does protect inner ear hair cells from the toxic effects of cisplatin.

### Example 3

This Example shows that ebselen, and the combination of ebselen and allopurinol, protect rat inner ear hair cells from damage by cisplatin in vivo.

Auditory Evoked Brainstem Response (ABR) was used to assess hearing in rats before and after exposure to cisplatin and chemoprotectants. Ebselen or DMSO (control vehicle) were introduced intraperitoneally into rats one hour before intraperitoneal administration of cisplatin at a dosage of 16 mg/kg body weight. Seventy-two hours after delivery of cisplatin, ABR data were collected, animals were sacrificed, cochleae were collected, dissected, stained with FITC-phalloidin (to detect F-Actin in hair cells), and DAPI (to detect nuclear DNA).

FIG. 6 shows the permanent threshold shift (PTS) in hearing, at 8 kHz, 16 kHz, 24 kHz and 32 kHz, of rats treated with cisplatin (at a dosage of 16 mg/kg body weight) in the presence of ebselen (at a dosage of 16 mg/kg body weight) (22), or in the presence of saline and DMSO (control) (20). Ten cochlea were tested per treatment. The PTS is a measure of hearing loss. The data presented in FIG. 6 show that the PTS is less (i.e., there is less hearing loss) in rats treated with the combination of ebselen and cisplatin, compared to rats treated with cisplatin without ebselen.

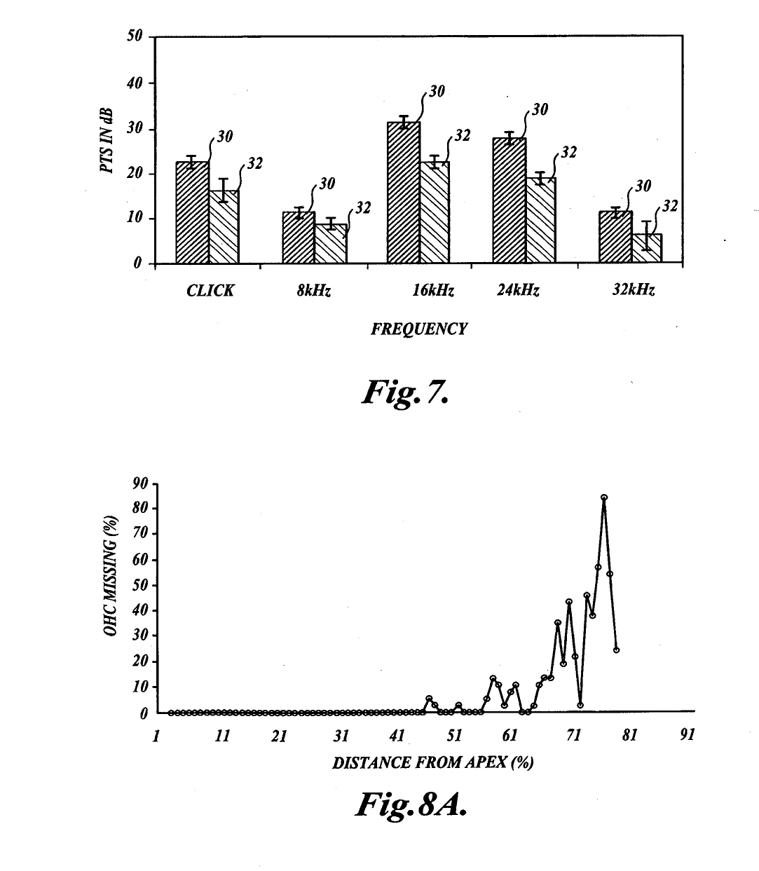

FIG. 7 shows the permanent threshold shift (PTS) in hearing, at 8 kHz, 16 kHz, 24 kHz and 32 kHz, of rats treated with cisplatin (at a dosage of 16 mg/kg body weight) in the presence of allopurinol (at a dosage of 16 mg/kg body weight) (30), or in the presence of the combination of allopurinol (at a dosage of 8 mg/kg body weight) and ebselen (at a dosage of 8 mg/kg body weight) (32). Four cochlea were tested per treatment. The data presented in FIG. 7 show that the PTS is less in rats treated with the combination of ebselen and allopurinol, compared to rats treated with allopurinol without ebselen.

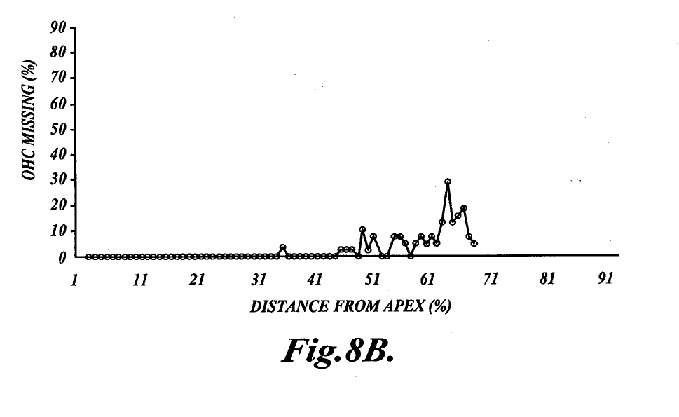

Additionally, cochleae were excised from rats treated with the combination of cisplatin and ebselen as described in this Example. Cochleae were also excised from rats treated with cisplatin and saline and DMSO (control). The number of outer auditory hair cells in the excised cochlea were counted at intervals of 0.1 mm along the cochlea. Representative results from a control rat and a treated rat are shown in FIG. 8A and FIG. 8B, respectively. The data presented in FIG. 8A and FIG. 8B show that the percentage of outer hair cells missing in cochleae from rats treated with the combination of cisplatin and ebselen is less than the percentage of outer hair cells missing in cochleae from rats treated with cisplatin, but not with ebselen.

### Example 4

This Example shows that the combination of ebselen and allopurinol enhances the chemotherapeutic effect of cisplatin against the ovarian cancer cell line NuTu-19 that has been introduced into rats.

An ovarian cancer tumor model was established in rats by injection of 10⁷ NuTu-19 cells into the peritoneal cavity of 8-10 week old female F-344 rats. Rats with injected NuTu-19 cells were allowed to develop tumor burden for two weeks prior to cisplatin treatment. A control series of 10 rats was evaluated separately for the development of ovarian tumor burden under the described conditions. All rats in the control series were sacrificed 5 weeks after NuTu-19 tumor cell injection and tumor burden was evaluated. In this control series, all animals exhibited significant tumor burden exemplified by omental caking of multiple tumor nodules and large volumes of ascites (10-30 mL) in the peritoneal cavity.

The response of the NuTu-19 tumors to cisplatin, in the presence or absence of the combination of ebselen and allopurinol, was also assessed. The presence of ascites and omental tumor caking was considered to be an indication that the cancer did not respond to the treatment. The absence of ascites, but presence of more than 5 visible tumor nodules (each >0.5 mm) in the peritoneal cavity was considered to be an indication that the cancer partially responded to the treatment. The absence of ascites and presence of fewer than 5 visible tumor nodules (each >0.5 mm) was considered to be an indication that the cancer fully responded to the treatment. The results of these experiments are shown in Table 1.

TABLE 1

| | NuTu-19 | |
|---|---|---|
| Tumor Response | Cisplatin | Cisplatin + ebselen and allopurinol |
| % Complete | 57 | 92 |
| % Partial | 43 | 8 |
| % No | 0 | 0 |

The following abbreviations are used in Table 1: “% Complete” refers to rats that showed no sign of tumor burden; “% Partial” refers to rats that showed some evidence of tumor burden; and “% No” refers to rats that had tumors that were not responsive to cisplatin treatment.

Results: The ovarian epithelial carcinoma cell NuTu-19 is syngeneic for the Fischer 344 rat, and is recognized as a clinically relevant model for ovarian cancer. See, e.g., Rose, G. S., et al. Am. J. Obstet. Gynecol., 175:593-599, 1996; Cloven, N. G., et al., Anticancer Res., 20(6B):4205-9, 2000; and Stakleff et al., Int. J. Gynecol. Cancer, 15:246-254, 2005. After injection into a Fischer 344 rat, NuTu-19 cells cause aggressive and highly metastatic tumors that are generally responsive to cisplatin treatment (see Lynch et al., Anti-Cancer Drugs, 16:569-579, 2005).

The results shown above in TABLE 1 indicate that the combined formulation of ebselen and allopurinol produced no inhibitory effect on cisplatin's anti-tumor activity, and in fact enhanced the efficacy of cisplatin in the NuTu-19 ovarian cancer tumor model.

### Example 5

This Example shows that ebselen and the combination of ebselen and allopurinol possess chemotherapeutic activity when administered to mammalian ovarian cancer cell lines. This Example also shows that ebselen and the combination of ebselen and allopurinol act to enhance the chemotherapeutic activity of platinum-containing chemotherapeutic agents.

Ovarian Cancer Cell Lines Tested:

ES-2 (human clear cell carcinoma) multi-drug chemotherapy resistance

SKOV-3 (human adenocarcinoma) multi-drug chemotherapy resistance

OVCAR-3 (human adenocarcinoma) multi-drug chemotherapy resistance

CAOV-3 (human adenocarcinoma)

OV-90 (human mixed morphology: papillary serous adenocarcinoma)

TOV-112D (human mixed morphology: adenocarcinoma/endometroid carcinoma)

TOV-21G (human mixed morphology: adenocarcinoma/clear cell carcinoma)

rSPI-tu-rat epithelial ovarian cancer cell line

Culture Conditions: Cells were kept in logarithmic growth and passed weekly or twice weekly. CAOV-3 was maintained in Dulbecco's modified Eagle's medium with 4.5 g/L glucose, and 10% fetal bovine serum (FBS). OVCAR-3 was maintained in RPMI 1640 medium with 2 mM 1-glutamine, 1.5 g/L sodium bicarbonate, 4.5 g/L glucose, 10 mM HEPES, 1 mM sodium pyruvate, 0.01 mg/mL bovine insulin, and 20% FBS. SKOV-3 and ES-2 were maintained in a 1:1 mixture of MCDB 105 medium and medium 199, with 15% FBS. OV-90 was maintained in a 1:1 mixture of MCDB 105 medium and medium 199 with 10% FBS.

IC₅₀ Inhibition Assay for Paclitaxel and Cisplatin

Prior to the initiation of the cytotoxicity study, growth inhibition assays were carried out to determine the IC₅₀ concentration for incubation of each ovarian cell line in the presence of paclitaxel and cisplatin for 96 hours, as shown below in TABLE 2.

TABLE 2
IC₅₀ Concentrations for Paclitaxel and Cisplatin

| Paclitaxel | | | | | | | | |
|---|---|---|---|---|---|---|---|---|
| plus | | | | | | | | |
| cisplatin | ES-2 | SKOV3 | OVCAR3 | CAOV-3 | OV90 | TOV112D | TOV21G | rSPI-tu |
| Paclitaxel | 7.2 | 10.0 | 1.8 | 1.76 | 38.5 | 2.6 | 80 | 90 |
| (nM) | | | | | | | | |
| Cisplatin | 4.0 | 4.4 | 1.4 | 1.4 | 4.4 | 1.05 | 4.8 | 1.5 |
| (μM) | | | | | | | | |

Results of IC₅₀ analysis: In the combined treatment with paclitaxel and cisplatin, the IC₅₀ concentration of paclitaxel ranged from 1.9 nM to 90 nM and the concentration of cisplatin ranged from 1.4 μM to 4.8 μM.

Cytotoxicity Study with Ebselen and Allopurinol

Each of the cell lines listed above was plated at a density of 3,500 cells per well in 96 well plates and incubated at 37° C. for 24 hours. Each of the cell lines was treated with 50 μl of either ebselen, allopurinol, or ebselen plus allopurinol at concentrations ranging from 0 to 100 μM and incubated for one hour. After the one hour incubation, 50 μl of cisplatin and paclitaxel were added to the cell lines at the various concentrations shown in TABLE 2 to achieve an IC₅₀ in the absence of other drugs. The cells were then incubated at 37° C. in 5% CO₂ in the presence of the cisplatin and paclitaxel for an additional 67-72 hours. Control wells were included as follows: no drug, ebselen, allopurinol, ebselen and allopurinol, and cisplatin/paclitaxel.

After the 67-72 hour incubation period, 10 μl of MTT (3-(4,5-dimethlythiazol-2-yl)-2,5-diphenyl tetrasodium bromide) working solution was added to each well (based on the instructions in the Chemicon MTT Cell Growth Kit Cat #CT01) and the cells were incubated for 4 hours. 100 μl of isopropanol/HCl solution was then added to each well and absorbance at 570 nm was measured. The results of the MTT assay for the different ovarian cancer cell lines tested are shown in FIGS. 9A-16C and are summarized below in TABLE 3.

Results:

FIGS. 9A-16C show the results of the various cell lines incubated in the presence of ebselen, allopurinol, ebselen plus allopurinol, and paclitaxel plus cisplatin. The results shown in FIGS. 9A-16C are summarized below in TABLE 3. The results presented show that 1) ebselen possesses chemotherapeutic activity against ovarian cancer cell lines; 2) allopurinol does not reduce the chemotherapeutic activity of ebselen; and 3) ebselen enhances the chemotherapeutic activity of cisplatin plus paclitaxel.

Ebselen Possesses Chemotherapeutic Activity Against Ovarian Cancer Cell Lines.

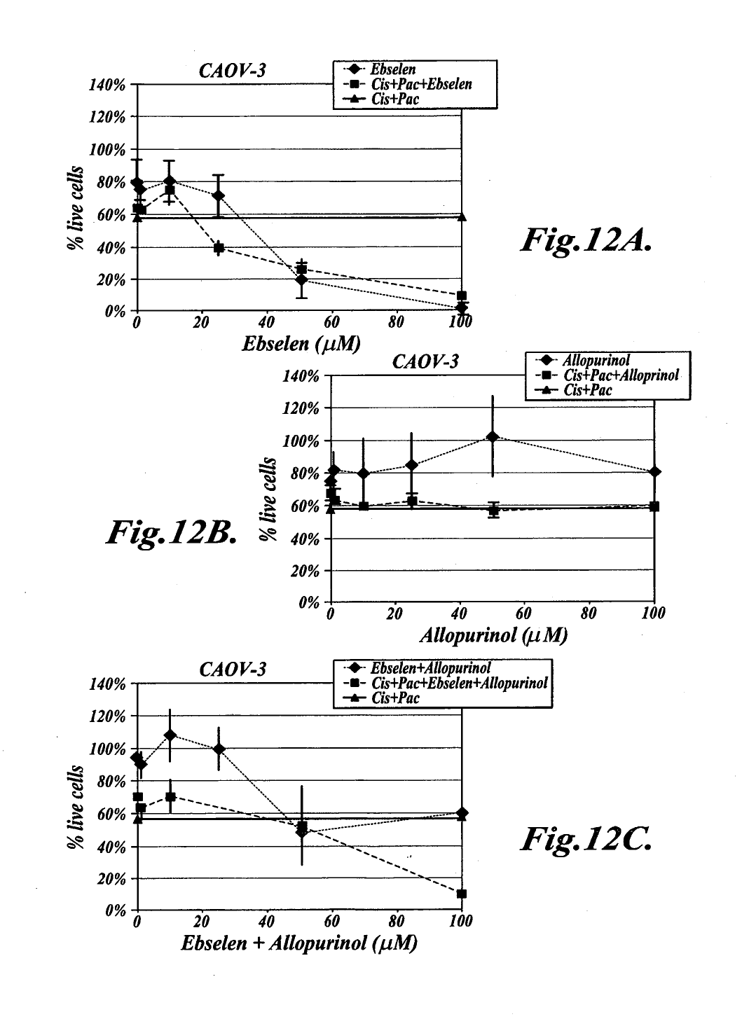

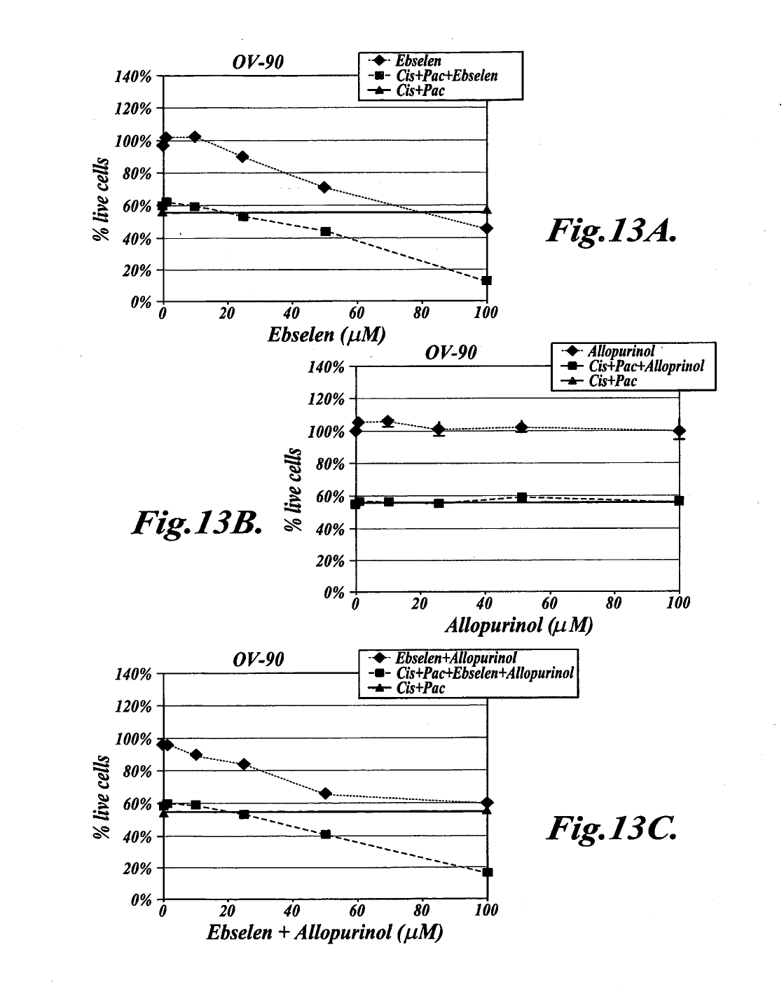

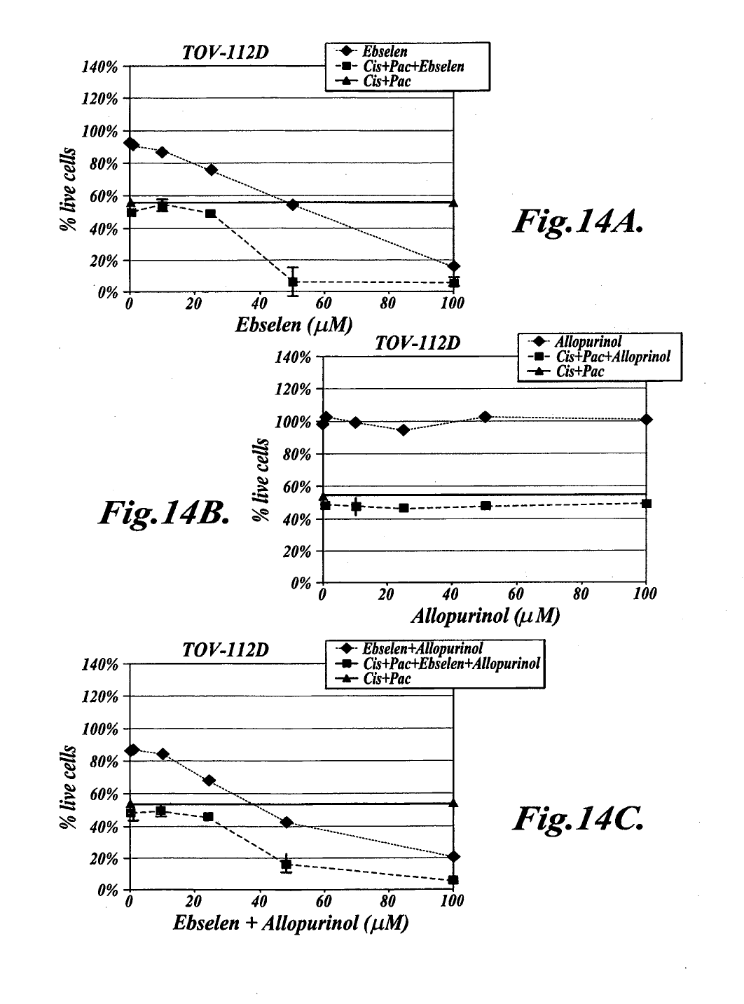

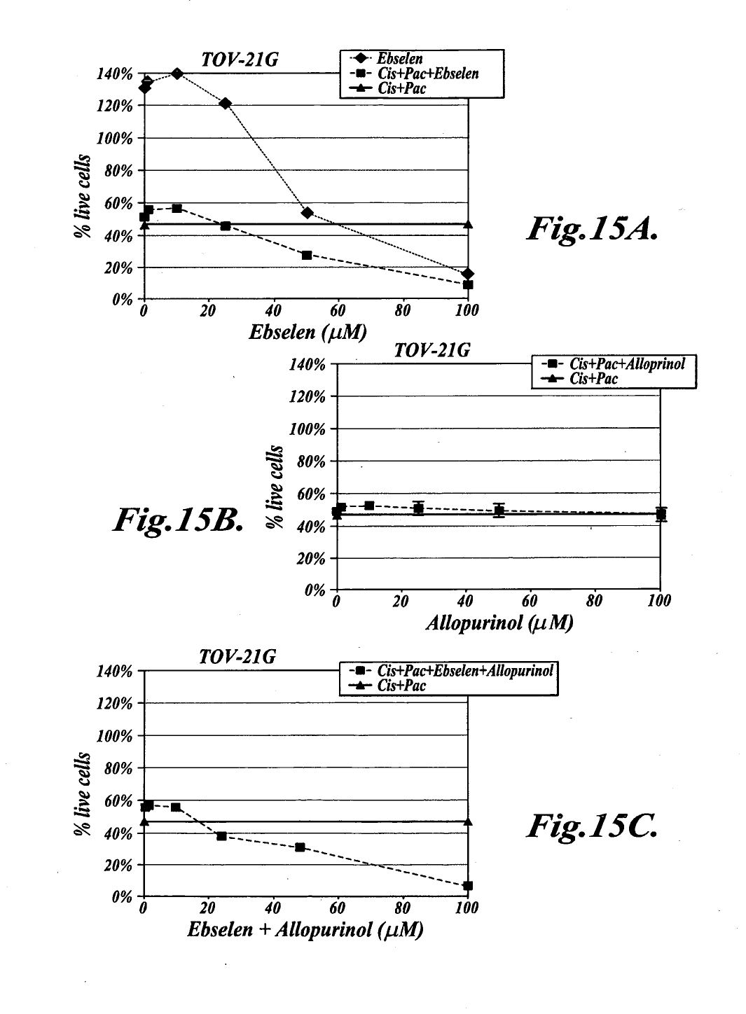

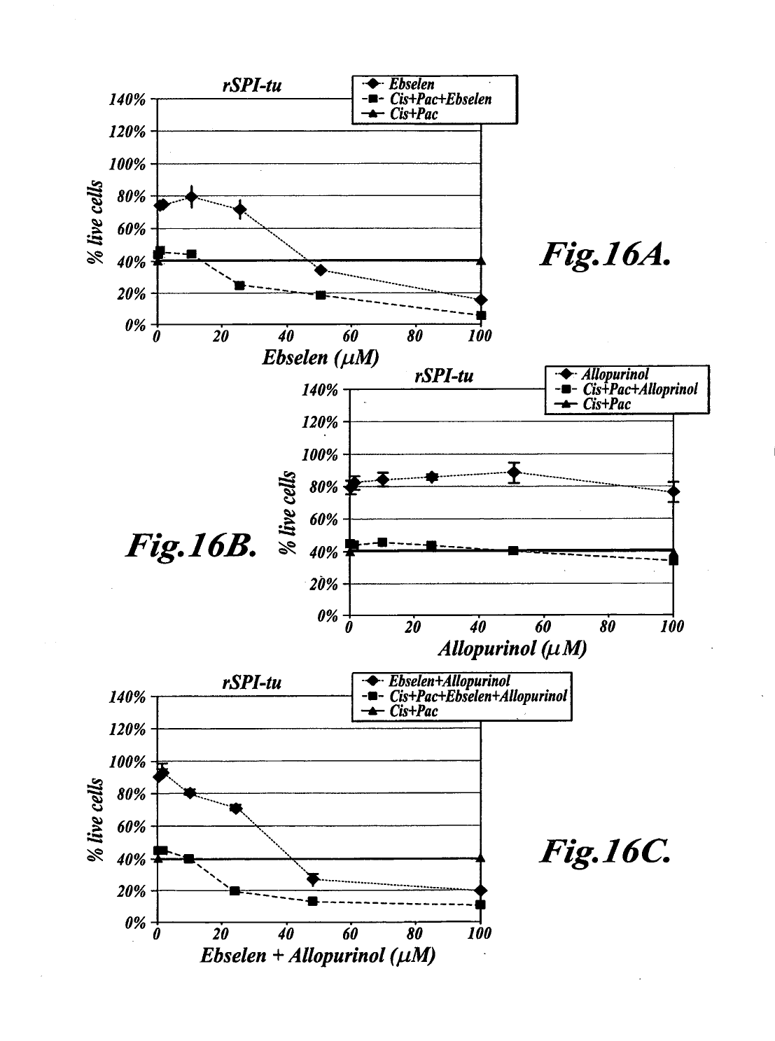

As summarized in TABLE 3, ebselen acts as a chemotherapeutic agent on all of the seven mammalian ovarian cancer cell lines tested, which include ES-2 (FIG. 9A), SKOV-3 (FIG. 10A), OVCAR-3 (FIG. 11A), CAOV-3 (FIG. 12A), OV-90 (FIG. 13A), TOV-112D (FIG. 14A), TOV-21G (FIG. 15A) and rSPI-tu (FIG. 16A). In all cell lines tested, ebselen induced dose-dependant cytotoxicity in the concentration range from 20 μM to 100 μM. In contrast, as shown in FIG. 5, ebselen does not appear to have a cytotoxic effect on mouse cochlear inner ear cells at 47 μM. Allopurinol did not have a toxic effect on any of the mammalian ovarian cancer cell lines tested, in a concentration range of from 20 μM to 100 μM, as shown in TABLE 3, TABLE 4 and FIGS. 9B, 10B, 11B, 12B, 13B, 14B, 15B and 16B. Moreover, when allopurinol was present in combination with ebselen, allopurinol did not reduce the chemotherapeutic effect of ebselen, as shown in TABLE 3, TABLE 4 and FIGS. 9C, 10C, 11C, 12C, 13C, 14C, 15C and 16C.

Ebselen and the Combination of Ebselen and Allopurinol Enhances the Chemotherapeutic Effect of Cisplatin Plus Paclitaxel Against Ovarian Cancer Cell Lines

As summarized in TABLE 4 below, and shown in FIGS. 9A-16C, ebselen and the combination of ebselen and allopurinol in the concentration range from 20 μM to 100 μM enhances the chemotherapeutic effect of cisplatin plus paclitaxel against all of the seven mammalian ovarian cancer cell lines tested, which include ES-2 (FIG. 9C), SKOV-3 (FIG. 10C), OVCAR-3 (FIG. 11C), CAOV-3 (FIG. 12C), OV-90 (FIG. 13C), TOV-112D (FIG. 14C), TOV-21G (FIG. 15C) and rSPI-tu (FIG. 16C).

Moreover, as shown in TABLE 4 below, ebselen and the combination of ebselen and allopurinol enhanced the chemotherapeutic activity of cisplatin and paclitaxel in each of the multi-drug chemotherapy resistant cell lines tested, which include ES-2 (FIGS. 9A, 9C), SKOV-3 (FIGS. 10A, 10C) and OVCAR-3 (FIGS. 11A, 11C).

TABLE 3
Percent live cells after 96 hour drug treatment

| Drug | ES-2 | SKOV-3 | OVCAR-3 | CAOV-3 | OV-90 | TOV-112D | TOV-21G | rSPI-tu |
|---|---|---|---|---|---|---|---|---|
| ebselen | ~20% | ~40% | ~20% | ~0% | ~40% | ~18% | ~18% | ~20% |
| (100 μM) | | | | | | | | |
| allopurinol | ~90% | ~90% | ~100% | ~80% | ~100% | ~100% | N.D. | ~80% |
| (100 μM) | | | | | | | | |
| ebselen | ~40% | ~40% | ~15% | ~60% | ~60% | ~20% | N.D. | ~20% |
| (100 μM) + | | | | (+/−20%) | | | | |
| allopurinol | | | | | | | | |
| (100 μM) | | | | | | | | |
| cisplatin + | ~70% | ~60% | ~80% | ~60% | ~55% | ~55% | ~45% | ~40% |
| paclitaxel | (cis4 μM/ | (cis4.4 μM/ | (cis1.4 μM/ | (cis1.4 μM/ | (cis4.4 μM/ | (cis1.05 μM/ | (cis4.8 μM/ | (cis:1.5 μM/ |
| | pac 7.2 nM) | pac 10 nM) | pac 1.8 nM) | pac 1.76 nM) | pac 38.5 nM) | pac: 2.6 nM) | pac 80 nM) | pac 90 nM) |

TABLE 4
Percent live cells after 96 hour drug treatment with cisplatin plus paclitaxel

| | ES-2 | SKOV-3 | OVCAR-3 | CAOV-3 | OV-90 | TOV-112D | TOV-21G | rSPI-tu |
|---|---|---|---|---|---|---|---|---|
| | (cis4 μM/ | (cis4.4 μM/ | (cis1.4 μM/ | (cis1.4 μM/ | (cis4.4 μM/ | (cis1.05 μM/ | (cis4.8 μM/ | (cis:1.5 μM/ |
| Drug | pac 7.2 nM) | pac 10 nM) | pac 1.8 nM) | pac 1.76 nM) | pac 38.5 nM) | pac: 2.6 nM) | pac 80 nM) | pac 90 nM) |
| cisplatin + | ~70% | ~60% | ~80% | ~60% | ~55% | ~55% | ~45% | ~40% |
| paclitaxel | | | | | | | | |
| ebselen | ~5.0% | ~10% | ~5% | ~10% | ~10% | ~5.0% | ~10% | ~20% |
| (100 μM) | | | | | | | | |
| plus | | | | | | | | |
| (cisplatin + | | | | | | | | |
| paclitaxel) | | | | | | | | |
| allopurinol | ~80% | ~65% | ~100% | ~80% | ~100% | ~100% | ~55% | ~80% |
| (100 μM) | | | | | | | | |
| plus | | | | | | | | |
| (cisplatin + | | | | | | | | |
| paclitaxel) | | | | | | | | |
| (ebselen | ~5.0% | ~10% | ~10% | ~10% | ~18% | ~5.0% | ~5.0% | ~20% |
| (100 μM) + | | | | | | | | |
| allopurinol | | | | | | | | |
| (100 μM)) | | | | | | | | |
| plus | | | | | | | | |
| (cisplatin + | | | | | | | | |
| paclitaxel) | | | | | | | | |

While the preferred embodiment of the invention has been illustrated and described, it will be appreciated that various changes can be made therein without departing from the spirit and scope of the invention.

1. A method for treating cancer in a mammal, the method comprising the step of administering to a mammal suffering from a cancer an amount of ebselen sufficient to inhibit the growth of the cancer.

2. The method of claim 1 wherein the mammal is a human being.

3. The method of claim 1 wherein the cancer is ovarian cancer.

4. The method of claim 1 wherein the cancer is testicular cancer.

5. The method of claim 1 wherein the cancer is a cancer of the head or neck.

6. The method of claim 1 wherein the cancer exhibits multi-drug chemotherapy resistance.

7. The method of claim 1 wherein the ebselen is administered in an amount of from 5 to 5000 mg/day.

8. The method of claim 1 wherein the ebselen is administered periodically to the mammal over a time period of from one month to 24 months.

9. The method of claim 1 wherein the ebselen is administered once per day to the mammal over a time period of from one month to 24 months.

10. The method of claim 1 wherein the mammal is not administered another chemotherapeutic agent in addition to ebselen.

11. A method for treating cancer in a mammal, the method comprising the step of administering to a mammal suffering from a cancer an amount of ebselen and an amount of allopurinol sufficient to inhibit the growth of the cancer.

12. The method of claim 11 wherein the mammal is a human being.

13. The method of claim 11 wherein the cancer is ovarian cancer.

14. The method of claim 11 wherein the cancer is testicular cancer.

15. The method of claim 11 wherein the cancer is a cancer of the head or neck.

16. The method of claim 11 wherein the cancer exhibits multi-drug chemotherapy resistance.

17. The method of claim 11 wherein the allopurinol is administered in an amount of from 10 to 2400 mg/day, and the ebselen is administered in an amount of from 5 to 5000 mg/day.

18. The method of claim 11 wherein the allopurinol and ebselen are administered periodically to the mammal over a time period of from one month to 24 months.

19. The method of claim 11 wherein the allopurinol and ebselen are administered once per day to the mammal over a time period of from one month to 24 months.

20. The method of claim 11 wherein the mammal is not administered another chemotherapeutic agent in addition to ebselen and allopurinol.

21. A method for enhancing the chemotherapeutic effect of a platinum-containing chemotherapeutic agent administered to a mammal suffering from cancer, the method comprising the step of administering to a mammal suffering from cancer an amount of 2-phenyl-1,2-benzoisoselenazol-3(2H)-one sufficient to enhance the chemotherapeutic effect of a platinum-containing chemotherapeutic agent on the cancer, wherein the 2-phenyl-1,2-benzoisoselenazol-3(2H)-one is administered to the mammal before, during or after administration of the chemotherapeutic agent to the mammal.

22. The method of claim 21 wherein the cancer is a cancer of the female reproductive system.

23. The method of claim 21 wherein the cancer is ovarian cancer.

24. The method of claim 21 wherein the cancer is testicular cancer.

25. The method of claim 21 wherein the cancer is a cancer of the head or neck.

26. The method of claim 21 wherein the cancer exhibits multi-drug chemotherapy resistance.

27. The method of claim 21 wherein a taxane containing chemotherapeutic agent is also administered to the mammal.

28. The method of claim 21 wherein the platinum-containing chemotherapeutic agent is selected from the group consisting of cisplatin and carboplatin.

29. The method of claim 27 wherein the taxane containing chemotherapeutic agent is selected from the group consisting of paclitaxel and docetaxel.

30. The method of claim 21 wherein the 2-phenyl-1,2-benzoisoselenazol-3(2H)-one is administered in an amount of from 5 to 5000 mg/day.

31. A method for enhancing the chemotherapeutic effect of a platinum-containing chemotherapeutic agent administered to a mammal suffering from cancer, the method comprising the step of administering to a mammal suffering from cancer an amount of allopurinol and an amount of 2-phenyl-1,2-benzoisoselenazol-3(2H)-one sufficient to enhance the chemotherapeutic effect of a platinum-containing chemotherapeutic agent on the cancer, wherein the allopurinol and 2-phenyl-1,2-benzoisoselenazol-3(2H)-one are administered to the mammal before, during or after administration of the chemotherapeutic agent to the mammal.

32. The method of claim 31 wherein the cancer is a cancer of the female reproductive system.

33. (canceled)

33. (canceled)

34. The method of claim 31 wherein the cancer is a cancer of the head or neck.

35. The method of claim 31 wherein the cancer exhibits multi-drug chemotherapy resistance.

36. The method of claim 31 wherein a taxane containing chemotherapeutic agent is also administered to the mammal.

37. The method of claim 31 wherein the platinum-containing chemotherapeutic agent is selected from the group consisting of cisplatin and carboplatin.

38. The method of claim 31 wherein said allopurinol is administered in an amount of from 10 to 2400 mg/day, and said 2-phenyl-1,2-benzoisoselenazol-3(2H)-one is administered in an amount of from 5 to 5000 mg/day.

39. A method of ameliorating at least one adverse effect of a platinum-containing chemotherapeutic agent, the method comprising the step of administering to a mammal suffering from cancer an amount of allopurinol and an amount of 2-phenyl-1,2-benzoisoselenazol-3(2H)-one sufficient to ameliorate at least one adverse effect of the platinum-containing chemotherapeutic agent, wherein the allopurinol and 2-phenyl-1,2-benzoisoselenazol-3(2H)-one are administered to the mammal before, during or after administration of the chemotherapeutic agent to the mammal.

40. The method of claim 39, wherein the platinum-containing chemotherapeutic agent is cisplatin.

41. The method of claim 31 wherein the cancer is ovarian cancer.

42. The method of claim 31 wherein the cancer is testicular cancer.
